(19) United States
(12) Patent Application Publication
(10) Pub. No.: US 2016/0199242 A1
(43) Pub. Date: Jul. 14, 2016

(54) EXTREMITY SURGICAL SUPPORT DEBRIDEMENT PLATFORM

(71) Applicant: Christopher T. Born, Providence, RI (US)

(72) Inventor: Christopher T. Born, Providence, RI (US)

(21) Appl. No.: 15/077,709

(22) Filed: Mar. 22, 2016

Related U.S. Application Data
(63) Continuation-in-part of application No. 14/463,729, filed on Aug. 20, 2014.

(51) Int. Cl. A61G 13/10; A61G 13/12; A61M 1/00

(52) U.S. Cl. CPC A61G 13/102; A61M 1/0056; A61M 1/008; A61G 13/12

(57) ABSTRACT

A platform for debridement of an extremity includes a body member having a bottom and a side wall to define an interior for collecting fluid used to wash the extremity. The side wall defines a drainage aperture for receiving a suction tube for removing the fluid from the interior. A cover defines drainage holes and secures to the body member to enclose the interior volume. Support members upstand from the bottom for supporting the cover. The support members follow the contour of the cover. A suction pad removes fluid from the interior. The suction pad includes a central disc having an upper surface defining a central port for connection to a suction hose and a sidewall a plurality of radial inlets in fluid communication with the central port. A porous material covers the plurality of radial inlets.

### CROSS REFERENCE TO RELATED APPLICATIONS

This continuation-in-part application is related to and claims priority from earlier filed, U.S. Non-Prov. application Ser. No. 14/463,729 filed Aug. 20, 2014, which is a continuation-in-part of U.S. Non-Prov. application Ser. No. 13/844,430 filed Mar. 15, 2013, which is a continuation-in-part of U.S. Non-Prov. application Ser. No. 12/837,144 filed Jul. 15, 2010, now issued U.S. Pat. No. 8,434,491, and U.S. Provisional Patent Application No. 61/225,845 filed Jul. 15, 2009, each of which is incorporated herein by reference.

### BACKGROUND

The present disclosure generally relates to surgical accessories and more particularly to a device that would allow support of an extremity during surgical and debridement procedures by medical personnel. More specifically, the present disclosure relates to an extremity surgical support debridement platform and suction device used to facilitate the process of debridement and lavage of injured extremities.

Surgical accessories adopted to allow medical personnel to perform medical treatment on a patient or a portion of a patient while collecting waste fluid or the like generated from such treatment are known. The most common type being a simple basin or towel. Other more complicated and expensive accessories are also known.

For example, U.S. Pat. No. 561,514 shows a bandaging table with a single liquid permeable surface containing several cutouts which receive medical instruments or other items required during the bandaging procedure. U.S. Pat. No. 2,609,261 shows a collapsible limb support which includes a screen surface with access to a drip pan. U.S. Pat. No. 2,945,731 shows a surgical drainage attachment for use with a standard operating table. This attachment comprises of a basin with a screen on top thereof for support of the hand or foot of a patient.

Further, U.S. Pat. No. 3,328,024 shows a drainage tray more or less permanently attached to a standard operating table having an upper screen surface and a lower drain opening. U.S. Pat. No. 4,082,257 shows a surgery table for use on a hand and includes a downwardly sloped upper operating surface with multiple drain holes positioned above a drain pan which includes a hose for removal of liquid wastes. U.S. Pat. No. 4,243,214 shows a high rectangular pan with multiple operating surfaces formed by moveable grids over a drip pan with a padded cutout for the patient's extremity.

It is always recommended to debride and wash out traumatic wounds and infections or to irrigate elective surgical sites of either the upper or lower extremities in either the operating room or the emergency room. This process always includes the use of saline irrigation in amounts that may vary from a few milliliters to several liters. The prior art does not disclosed a dedicated device into which the fluid can drain while the limb is uniformly supported as this debridement and lavage process takes place. The result is that this fluid frequently will pool on the operating room table or floor adding both a contamination risk and a fall hazard to the surgical environment.

The alternative technique that is employed is to use receptacles that are not dedicated to this process but which are commonly available in the surgical or emergency room suites. These often include ring or emesis basins and even bed pans. The limbs are not uniformly supported so that fractures and injured soft tissues undergo further trauma. Alternatively, hospitals may customize steel pans by placing a drain at one end or on the bottom and may also create a grid to go over the pan. These devices are often of insufficient length and/or too deep and prove to be cumbersome with the extremity having to be continually moved on and off the grid between the debridement and lavage components, thereby adding trauma to the tissues. The drains get clogged with tissue. These then have to be cleaned out and the suction re-attached all of which add delay to the procedure.

### SUMMARY

The disadvantages of the prior art are that they are cumbersome in size, not light in weight and not readily portable. They are not easily stackable for storage purposes and could not easily be included in pre-packaged sterile operating room or military-like field packs. Several require attachment to the operating room table or are too high to allow limb placement on the working surface without significant anatomical distortion of the extremity. Vascular and neural elements within the injured extremity can become kinked or torn as a result of these deformities. Several have high-riding side walls that will impede visualization and surgical access to the extremity during a procedure. The use of a fully circular hole or nozzle in the bottom or the sidewall for introduction or attachment of a drain hose in some makes insertion and removal difficult. Nozzles designed for drain hose attachment become easily clogged with tissue because of their narrow bore and frequently need to be cleared. The complicated nature of the prior art also lends to an increased expense for manufacturing.

Therefore, it is desirable for a medical device to facilitate the process of debridement and lavage of injured extremities that is easy to manipulate by a user, lightweight, portable, stackable, detached, durable, permits clear visualization of extremities, disposable and inexpensive to manufacture.

The present disclosure preserves the advantages of prior art for debridement platforms. In addition, it provides new advantages not found in currently available debridement platforms and overcomes many disadvantages of such currently available debridement platforms.

The present disclosure is generally directed to an extremity surgical support debridement platform. The debridement platform of the present disclosure enables the process of debridement and lavage of injured extremities that is easy to manipulate by a user, lightweight, portable, stackable, detached, durable, permits clear visualization of extremities, disposable and inexpensive to manufacture.

The debridement platform is dedicated to the process of debridement and lavage of injured extremities. The debridement profile is low profile, yet long and wide enough to uniformly support the entire limb for the entire debridement and lavage process. There are impediments to visualization or surgical access. Plastic suction tubing commonly used in hospitals is easily removed should it become clogged with tissue, but most of the debrided tissue would be captured by the overlying grid or cover member.

The platform generally includes a body member and a cover member. The subject technology consists of two pieces that include a shallow pan or body member of a generally rectangular shape having a liquid impervious body and an overlying removable, perforated horizontal work surface platform lid or cover member that are placed together under an extremity of a medical patient during the treatment of an extremity. The cover member and the body member are configured of flexible materials and relatively thin walls to cushion the extremity for comfort. It should be noted that the platform may have a one-piece configuration which is injection molded or two-piece or more configuration. The body member includes a bottom end and at least one side wall. The body member defines an interior volume for temporarily containing fluid.

At least one drainage aperture is defined within the body member for draining the fluid from the interior of the body member. The at least one drainage aperture generally defines a U shape or similar shape for easy removal of a drainage tube positioned therein. The at least one drainage aperture or side-entry portals are positioned proximal to a corner area of the body member where at least one side wall and bottom wall are joined together. The drainage aperture is positioned in lower portion of the sidewall substantially proximal to the bottom end of the body member. The draining apertures are incompletely circularized cutouts to allow the easy introduction of suction or drain hose for removal of fluid from the interior of the body member.

A cover member includes a top end and at least one side wall. The cover member and the body member are releasably engaged to define an overall low profile for easier deployment under an extremity and visibility of the extremity. The cover member defines at least one cover aperture for facilitating a flow of the fluid into interior of the body member. The cover member is perforated by a series of holes large enough to allow for the free flow of blood and other fluids into the underlying body member, yet small enough to prevent large pieces of tissue and other debris from falling into the body member during the procedure. The at least one cover aperture may have a smaller diameter within an extremity area where an extremity is resting on said cover member and a larger diameter outside said extremity area on said cover member to prevent pooling of fluid about the extremity.

The cover member may be configured and customized to accommodate a wide array of extremities for improved performance. The cover member has a low-profile splash guard positioned along an outer periphery of the cover member to prevent fluid from contacting a patient. The splash guard may be molded into the cover member. The cover member may be predefined with a recessed extremity area for providing further stability to extremity while seating on the cover member. Also, the top end of the cover member may have a slightly concave shape to prevent pooling of fluid about an extremity. Likewise, the body member may have a slightly convex shape to facilitate flow of fluid to the drainage apertures.

It is desirable to releasably engage the cover member and the body member which can be accomplished by a variety of methods. For example, a surface of said at least one side wall of the cover member is engaged to a surface of the at least one side wall of the body member to provide support and stability. The cover member may further include a rolled cover lip member for respectively engaging a rolled body lip member of the body member. The rolled cover lip member may nest within an inner surface of the rolled body lip member. Also, the rolled body lip member may nest within an inner surface of the rolled cover lip member. Alternatively, a lower periphery of the cover member may engage an upper periphery of the body member by a variety of methods, such as a friction fit, while still allowing for releasable engagement of the cover member to the body member.

At least one support member is positioned within interior of said body member. The cover member is supported from below by at least one support member or raised portions of the bottom end of the body member acting as uprights that are high enough to have contact with the cover member's undersurface. The at least one support member engages the body member and the cover member to provide stability and support. The at least one support member depends from bottom end of the body member. Alternatively, the at least one support member is defined within the body member in the shape of a tower. In one embodiment, a top portion of the at least one support member is keyed into the at least one cover aperture to prevent sheering of the cover member from the body member. The top portions of the at least one support member interdigits with corresponding at least one cover apertures in the cover member. The top portion of the at least one support member may include an upper region with a recessed shoulder region on either side of the upper region. The cover member seats down slightly into the body member, suspended around its periphery by at least one or more walls of the body member in addition to being supported from below by the at least one support members from the bottom end of the body member. This arrangement prevents side slippage of the cover member, yet allows for easy removal of the cover member during the procedure if required to clear debris or for insertion, removal or cleaning of the drainage tube.

At least one filtration trap may be positioned proximal to the at least one drainage aperture of said body member. To provide additional stability, the filtration trap has a lower profile than the side wall of the body member to engage a top portion of the filtration trap with the cover member for additional stability. In one embodiment, the filtration trap has a height approximately equal to the recessed shoulder region of the at least one support member. The filtration trap may be permanently or releasably attached to the body member. For example, the filtration trap may be clipped or inserted into a grooved area defined within the base member which can be removed for cleaning when clogged with debris.

Furthermore, an open cell material may be positioned proximal to the at least one cover aperture or the at least one draining aperture in the platform to prevent clogging of a drainage tube positioned within the at least one drainage aperture defined within the body member for draining the fluid from the interior of the body member. For example, the open cell material may be a sponge material or mesh material for absorbing the fluid.

A flexible strap member for securing the extremity to the platform may engage at least one cover aperture. The strap member includes a means for fastening at a proximal and distal ends of the strap member within the at least one cover aperture. The means for fastening at a proximal and distal ends of the straps may include fasteners or other devices known in the art. For example, a hook may be used to secure the proximal or distal end of the strap within the at least one cover aperture.

The cover member is sufficiently pliable for curling a portion of the cover member proximal to the at least one drainage aperture to provide access to the interior of the body member for removing clogging debris from the filtration trap and drainage tube while maintaining the extremity on the cover member. By locating the filtration trap, at least one U-shaped drainage aperture, and drainage tube in corner areas of the platform, a physician can more easily remove tube for cleaning and remove debris from the filtration traps.

The platform is made of material selected from a variety of materials including polycarbonate, HDPE (high density polyethylene), low density polyethylene, plastic materials, biodegradable materials, regrind materials, and recycled plastic materials in combination or alone. Also, the platform is injection molded using plastic materials to accommodate a wide range of extremity sizes and types at a lower cost.

A debridement surgical support kit for extremities of a human or mammal to assist a physician in treating an extremity may include the platform, as described herein, along with a surgery medical pack. The platform including the body member and the cover member. The cover member nested with the body member or the body member nested within the cover member to reduce the height of the platform to a lower profile for more efficient transport. The surgery medical pack including means for debridement and lavage of an extremity of a human known in the art such as surgical tools and other known surgical products for debridement and lavage. The surgery medical pack is positioned within an interior of the nested platform to facilitate transport and reduce overall profile. A box having a low profile may be used for containing the nested platform and surgery medical pack for purposes of transporting and storing said kit until required for surgery in a medical facility or storage area. In addition, one or more platforms may be stackable within one another to further reduce consumption of valuable space within a medical facility, especially a surgical environment.

In operation, the extremity is positioned upon an upper surface of the cover member to facilitate the process of debridement and lavage of extremities. The extremity is seated within the predefined recess area for additional support. The strap members are then secured over the top of the extremity within the at least one cover aperture. During cleaning and debridement, the fluid and travels through the at least one cover aperture for temporary storage within an interior of the body member. Next, the fluid passes through the filtration trap to capture any debris which could potentially clog the drainage tube. The fluid then exits the body member through the drainage tube positioned within the drainage aperture. If the drainage tube becomes clogged, a user curls or slightly opens a corner portion of the cover member to reveal a corner area of the body member where the filtration trap can be cleaned in place or removed for cleaning, the draining aperture can be cleaned, and the drainage tube can be removed. Most importantly, the extremity may continue to be positioned on the cover member, if desired, during the cleaning and unclogging of the drainage tube, filtration trap, and drainage aperture.

A portion of the body member may define perforations or cut-outs to facilitate removal of one or more sectional flap areas of the body member. For example, two opposing portions of the body member may be removed or hinged away to provide a defined path to position an extremity within the body member. The sectional flap areas may be hinged outward to remove the side wall portion of the body member. Alternatively, the sectional flap areas may be removed or turned inward to guide an extremity within an interior of the body member. In another embodiment, the body member may be pre-formed with the side wall portions of the body member removed in advance of use.

In one embodiment, the platform is made of material selected from a group consisting of: polycarbonate, HDPE (high density polyethylene), low density polyethylene, plastic materials, biodegradable materials, regrind materials, and recycled plastic materials in combination or alone. Preferably, the cover member has a low-profile splash guard positioned along an outer periphery of the cover member. The cover member and body member may be flexible materials to cushion the extremity for comfort.

It is therefore an object of the present disclosure to provide a platform that is easy to manipulate by a user and provides stability and support to the extremity. It is a further object of the embodiment to provide a platform that is stackable and easy to transport. Another object of the embodiment is to provide a platform that is disposable and inexpensive to manufacture.

Preferably, the platform permits clear visualization of extremities. Another object of the embodiment is to provide a platform that permits easy removal of the cover member during a surgical procedure to clear debris or for insertion, removal or cleaning of the drainage tube.

Other objects, features and advantages of the subject technology shall become apparent as the description thereof proceeds when considered in connection with the accompanying illustrative drawings.

### BRIEF DESCRIPTION OF THE DRAWINGS

The preferred embodiments of the subject technology, together with further objects and attendant advantages, will be best understood by reference to the following detailed description taken in connection with the accompanying drawings summarized below.

FIG. 1 is top view of the cover member of the described extremity surgical support debridement platform of the present disclosure.

FIG. 2 is a top view of the body member of the debridement platform as shown in FIG. 1.

FIG. 3A is an elevated side view of the cover member and body member of the debridement platform as shown in FIG. 1 without a drainage tube.

FIG. 3B is an elevated side view of the cover member and body member of the debridement platform as shown in FIG. 1 with a drainage tube.

FIG. 4A is an elevated side view of the cover member of the debridement platform as shown in FIG. 1 with a splash guard.

FIG. 4B is an elevated side view of the cover member of the debridement platform without a splash guard having a lower profile than FIG. 4A.

FIG. 5A is an elevated side view of the body member of the debridement platform as shown in FIG. 3A.

FIG. 5B is an elevated end view of the body member of the debridement platform as shown in FIG. 3A.

FIG. 6A is an elevated cross-sectional side view of the debridement platform as shown in FIG. 1 taken along line 6-6.

FIG. 6B is an elevated cross-sectional side view of another embodiment of the debridement platform as shown in FIG. 1 taken along line 6-6.

FIG. 7 is an elevated cross-sectional side view of the debridement platform as shown in FIG. 1 taken along line 7-7.

FIG. 8A is an elevated cross-sectional side view of another embodiment of debridement platform as shown in FIG. 6A where the cover member has a slight concave shape.

FIG. 8B is an elevated cross-sectional side view of another embodiment of debridement platform as shown in FIG. 6A where the body member has a slight convex shape.

FIG. 9A is partial cross-sectional view of another embodiment of the debridement platform as shown in FIG. 3A.

FIG. 9B is partial cross-sectional view of another embodiment of the debridement platform as shown in FIG. 3A.

FIG. 9C is partial cross-sectional view of another embodiment of the debridement platform as shown in FIG. 3A.

FIG. 10 is a top view of the debridement platform as shown in FIG. 1 with extremity recessed area defined therein.

FIG. 11 is a top view of the debridement platform as shown in FIG. 10 with strap members securing the extremity.

FIG. 12 is a perspective view of the debridement platform with the cover member being curled back for unclogging drainage tube.

FIG. 13 is a perspective view of the debridement platform in a kit.

FIG. 14 is an exploded view of another embodiment of the debridement platform.

FIG. 15 is an exploded view of still another embodiment of the debridement platform.

FIG. 16 is a perspective view of a suction pad used in conjunction with any debridement platform with the suction hose detached.

FIG. 17 is a perspective view of another embodiment of the debridement platform with perforated sectional flap areas.

FIG. 18 is a perspective of FIG. 17 with the sectional flap areas turned outward.

FIG. 19 is a top view of another embodiment of the debridement platform with a suction hose having a filtration component.

FIG. 20 is a partial top view of FIG. 19.

FIG. 21 is a top view of the suction hose and filtration assembly of FIG. 19.

FIG. 22 is a bottom view of the suction hose of FIG. 19 having the filtration component.

FIG. 23 is top view of the suction hose of FIG. 19 having an additional filter component in accordance with the subject technology.

FIG. 24 is a cut-away view of the suction hose of FIG. 19 having the filter component.

FIG. 25 is an exploded view of the filtration component of FIG. 19.

FIG. 26 is a top view of another filtration assembly attached to a suction hose in accordance with the subject technology.

### DETAILED DESCRIPTION OF THE PREFERRED EMBODIMENTS

In accordance with the present disclosure, an extremity surgical support debridement platform 10 is provided. The debridement platform enables the process of debridement and lavage of injured extremities that is easy to manipulate by a user, lightweight, portable, stackable, detached, durable, permits clear visualization of extremities, disposable and inexpensive to manufacture.

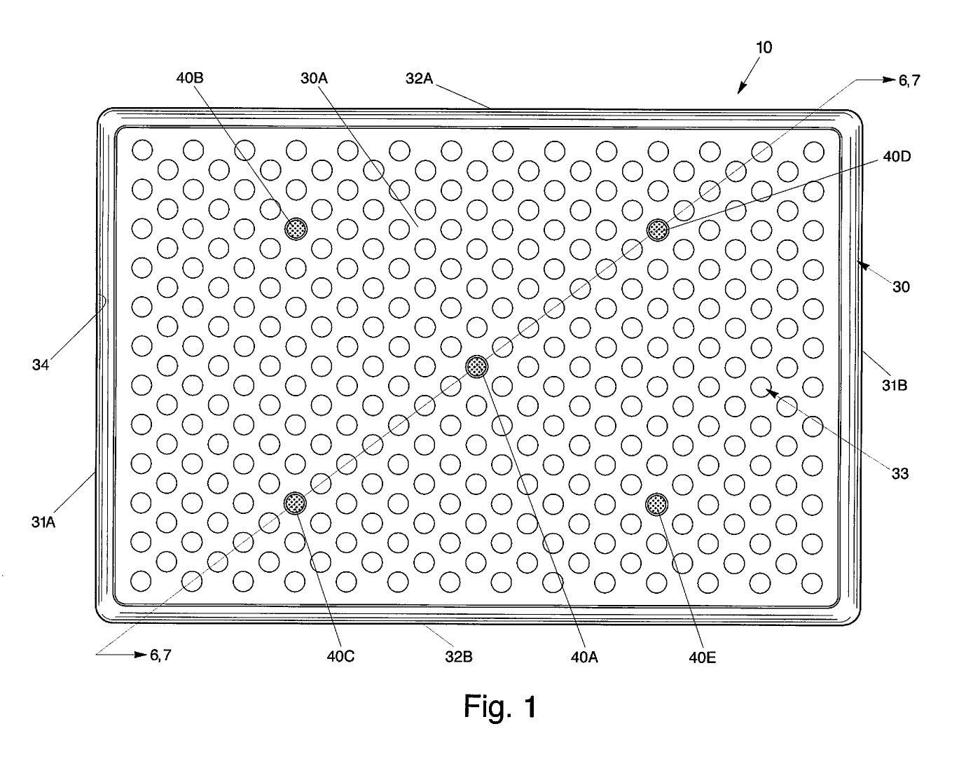

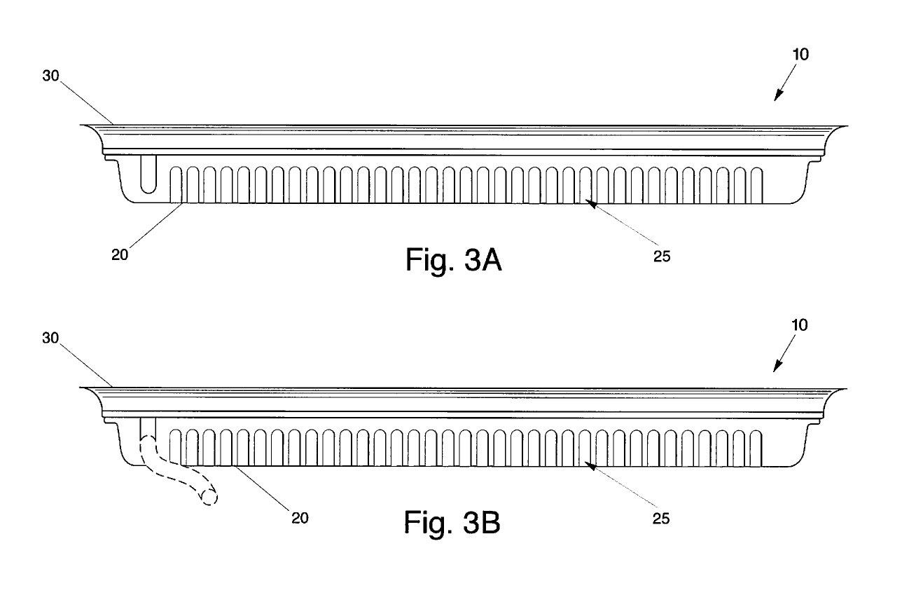

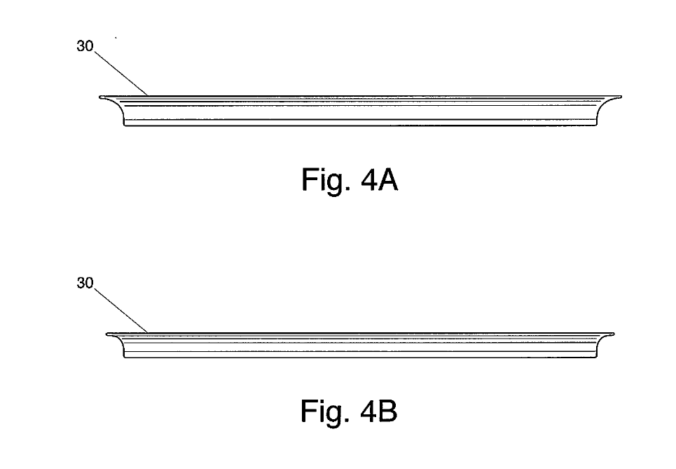

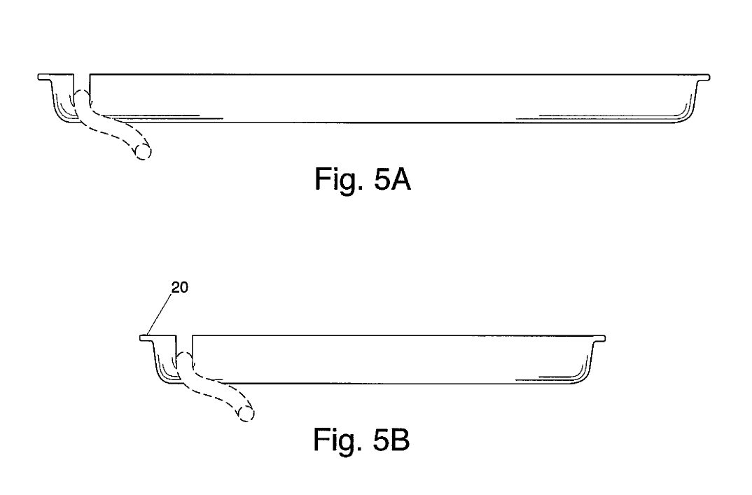

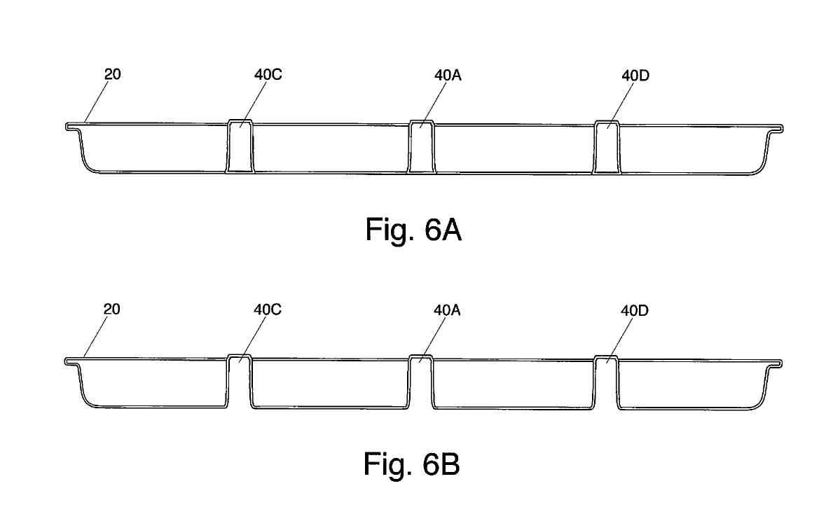

As shown generally in FIGS. 1-13, the debridement platform 10 is dedicated to the process of debridement and lavage of injured extremities. The key features are that it is low profile, yet long and wide enough to uniformly support the entire limb for the entire debridement and lavage process. There are impediments to visualization or surgical access. Plastic suction tubing commonly used in hospitals is easily removed should it become clogged with tissue, but most of the debrided tissue would be captured by the overlying grid or cover member 30.

The platform 10 generally includes a body member 20 and a cover member 30. The platform 10 consists of two pieces that include a shallow pan or body member 20 of a generally rectangular shape having a liquid impervious body and an overlying removable, perforated horizontal work surface platform lid or cover member 30 that are placed together under an extremity of a medical patient during the treatment of an extremity. The depth of the body member 20 is shallow enough to allow for easy placement under an injured limb without causing undue deformity or pressure but the cover member 30 is of appropriate surface dimensions to provide support. The cover member 30 and the body member 20 are configured of flexible materials and relatively thin walls to cushion the extremity for comfort. It should be noted that the platform 10 may have a one-piece configuration which is injection molded or two-piece or more configuration.

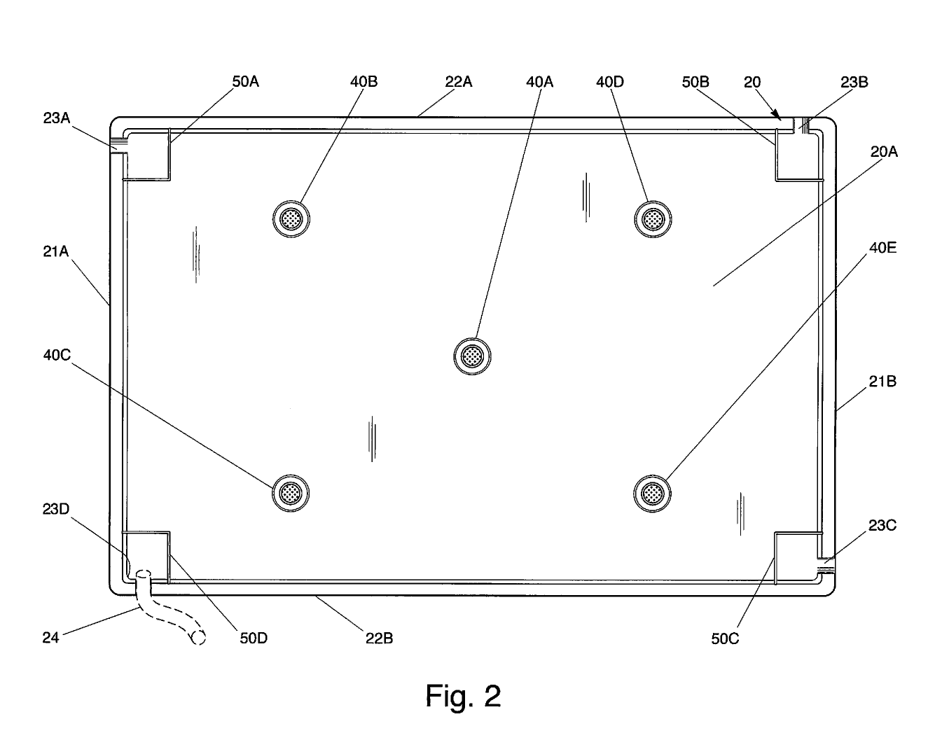

Referring to FIG. 2, the body member 20 includes a bottom end 20A and at least one side wall (21A, 21B, 22A, 22B). The body member 20 defines an interior volume for temporarily containing fluid excess from a surgical procedure or cleaning. In a preferred embodiment, the body member 20 includes a bottom end 20A and four side walls (21A, 21B, 22A, 22B). An outer surface of the four side walls (21A, 21B, 22A, 22B). may include a defined non-slip surface area 25 for better gripping by a user.

Still Referring to FIG. 2, at least one drainage aperture (23A, 23B, 23C, 23D) is defined within the body member 20 for placing a drainage tube 24 for draining the fluid from the interior of the body member 20. The at least one drainage aperture (23A, 23B, 23C, 23D) generally defines a U shape or similar shape for easy removal of the drainage tube 24 positioned therein. The U-shaped cutouts or drainage apertures (23A, 23B, 23C, 23D) at one end of the body member 20 are placed to allow a hose or drainage tube 24 to be passed into the bottom end 20A of the body member 20 to allow for fluid to be drained from the interior of the body member 20. This configuration means that the drainage tube 24 is merely laid into the “U” and does not have to be drawn through a hole or coupled to a nozzle. Of course, if desired, the drainage tube 24 may be connected coupled to a nozzle or drawn through a hole.

The at least one drainage aperture (23A, 23B, 23C, 23D) or side-entry portals are positioned proximal to a corner area of the body member 20 where at least one side wall (21A, 21B, 22A, 22B) and bottom end 20A are joined together. The drainage aperture (23A, 23B, 23C, 23D) is positioned in lower portion of the sidewall (21A, 21B, 22A, 22B) substantially proximal to the bottom end 20A of the body member 20. The draining apertures (23A, 23B, 23C, 23D) are incompletely circularized cutouts to allow the easy introduction of suction or drainage tube 24 for removal of fluid from the interior of the body member 20.

Referring to FIG. 1, a cover member 30 includes a top end 30A and at least one side wall (31A, 31B, 32A, 32B). In a preferred embodiment, the cover member 30 includes the top end 30A and four side walls (31A, 31B, 32A, 32B). The cover member 30 and the body member 20 are releasably engaged to define an overall low profile for easier deployment under an extremity and visibility of the extremity. In general, the cover member 30 is not typically permanently fixed to the underlying body member 20 and is designed for its easy removal, however, a single piece platform 10 may be used where the cover member 30 is permanently attached to the underlying body member 20. The cover member 30 is rectangular and approximately the same length and width as the body member 20.

The cover member 30 defines at least one cover aperture 33 for facilitating a flow of the fluid into an interior of the body member 20. The cover member 30 is perforated by a series of multiple cover apertures 33 large enough to allow for the free flow of blood, a brisk volume of irrigation fluid, and other fluids into the underlying body member 20, yet small enough to prevent large pieces of tissue and other debris from falling into the body member 20 during the procedure which may block or clog the drainage tube. The at least one cover aperture 33 may have a smaller diameter within a predefined recessed extremity area 35 where an extremity is resting on the cover member 30 and a larger diameter outside the extremity area on the cover member 30 to prevent pooling of fluid about the extremity. Also, the at least one cover aperture 33 may be chamfered to facilitate flow of fluid through the at least one cover aperture 33.

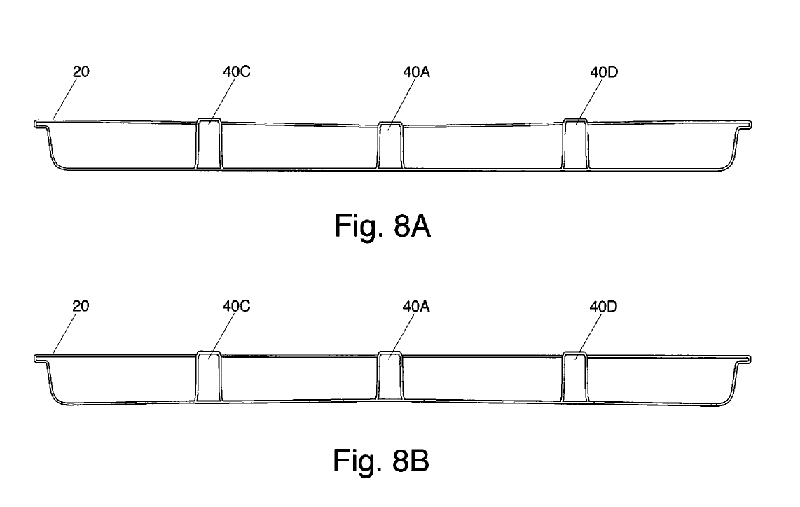

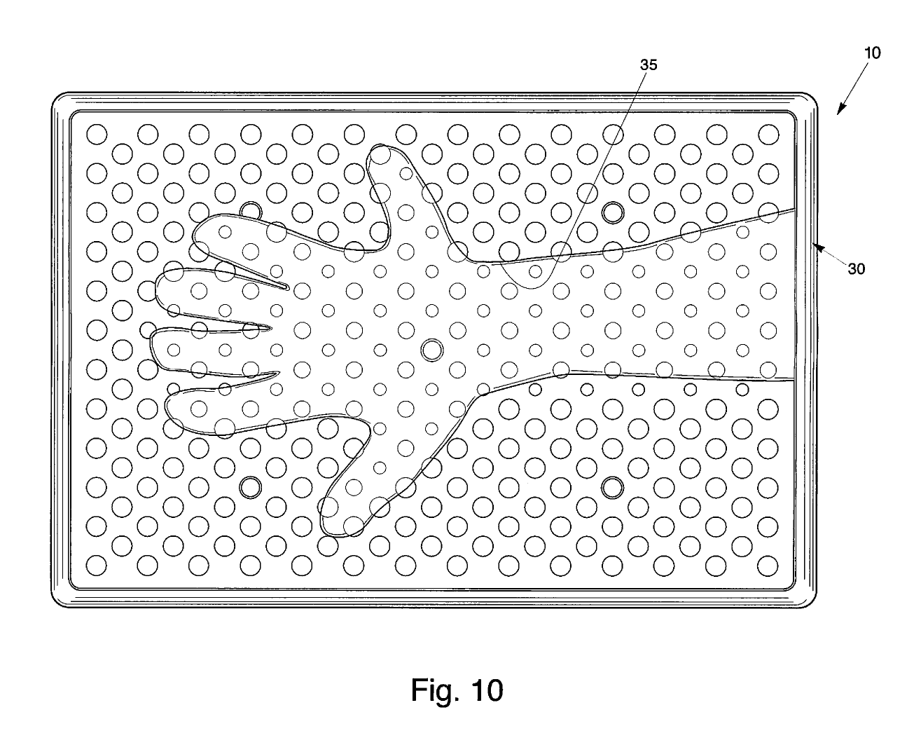

The cover member 30 may be configured and customized to accommodate a wide array of extremities for improved performance. The cover member 30 has a low-profile splash guard 34 positioned along an outer periphery of the cover member 30 to prevent fluid from contacting a patient. The cover member 30 may be predefined with a recessed extremity area 35 (FIG. 10) for providing further stability to extremity while seating on the cover member 30. Referring to FIG. 8A, the top end of the cover member 30 may have a slightly concave shape to prevent pooling of fluid about an extremity. Referring to FIG. 8B, the body member 20 may have a slightly convex shape to facilitate flow of fluid to the drainage apertures. The cover member 30 may also have molded-in channels (not shown) on a top surface of the cover member 30 to lead fluid to at least one cover aperture 33.

It is desirable to releasably engage the cover member 30 and the body member 20 which can be accomplished by a variety of methods. For example, a surface of the at least one side wall of the cover member 30 is engaged to a surface of the at least one side wall of the body member 20 to provide support and stability. A bottom edge of the cover member 30 may further include a rolled cover lip member for respectively engaging a top edge of the body member 20 having a rolled body lip member. Also, the rolled lip cover member 30 may be used for gripping by a user to facilitate handling of the debridement platform.

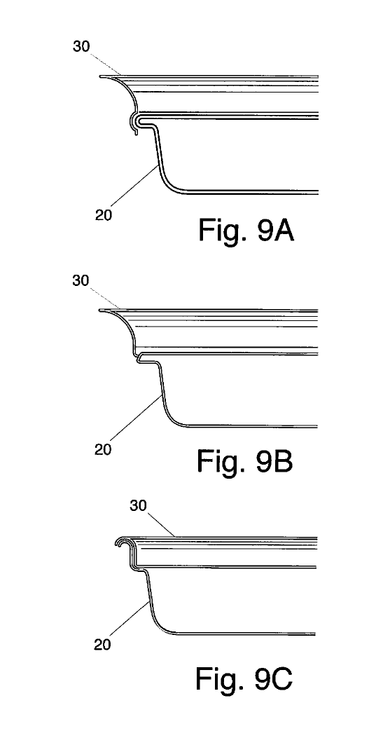

The edge of the body member 20 has a rolled lip member the undersurface of which matches the top surface of the edge of the underlying body member 20 to allow for nesting of the edge of the cover member 30 onto the edge of the body member 20. The top edge of the body member 20 is designed to have a rolled lip to accommodate the corresponding undersurface lip of an overlying cover member 30. The rolled cover lip member may nest within an inner surface of the rolled body lip member. Also, the rolled body lip member may nest within an inner surface of the rolled cover lip member. Alternatively, a lower periphery of the cover member 30 may engage an upper periphery of the body member 20 by a variety of methods, such as a friction fit, while still allowing for releasable engagement of the cover member 30 to the body member 20. FIGS. 9A-9C illustrates various configurations for attaching the body member 20 and cover member 30 together. In an alternative embodiment, the edge of the body member 20 and the edge of the cover member 30 are configured to allow them to snap together.

Referring to FIGS. 2-6, at least one support member (40A, 40B, 40C, 40D, 40E) is positioned within interior of the body member 20. For example, there are five support members (40A, 40B, 40C, 40D, 40E) arranged in specific configuration as illustrated in FIG. 2. Note, the at least one support member (40A, 40B, 40C, 40D, 40E) positioned in a central portion of the body member 20 may be slightly shorter in height than the other at least one support members (40A, 40B, 40C, 40D, 40E) to allow for contour of the cover member 30. The cover member 30 may be concave, convex, concave and convex, wavy and any desired shape to facilitate flow and/or collection of the fluid. The cover member 30 is supported from below by at least one support member (40A, 40B, 40C, 40D, 40E) or raised portions of the bottom end of the body member 20 acting as uprights that are high enough to have contact with the cover member's 30 undersurface. In one embodiment, the at least one support member (40A, 40B, 40C, 40D, 40E) is in the shape of a cylinder but it is contemplated that the at least one support member (40A, 40B, 40C, 40D, 40E) may be other shapes and sizes other than a cylinder such as a wall, pyramid, trapezoid, rectangle, arch and the like. The at least one support member (40A, 40B, 40C, 40D, 40E) engages the body member 20 and the cover member 30 to provide stability and support. The at least one support member (40A, 40B, 40C, 40D, 40E) depends or arises from the bottom end of the body member 20. Alternatively, the at least one support member (40A, 40B, 40C, 40D, 40E) is defined within the body member 20 in the shape of a tower.

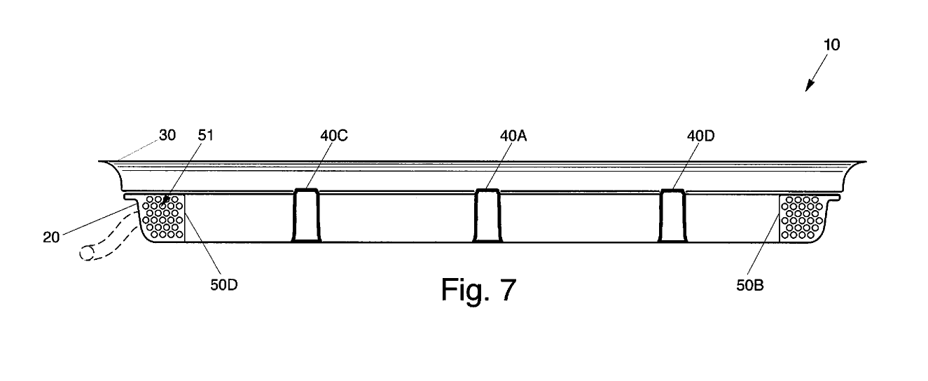

Referring to FIG. 7, in one embodiment, a top portion of the at least one support member (40A, 40B, 40C, 40D, 40E) is keyed into the at least one cover aperture 33 to prevent sheering of the cover member 30 from the body member 20. The top portions of the at least one support member (40A, 40B, 40C, 40D, 40E) interdigitates with corresponding at least one cover aperture 33s in the cover member 30. The top portion of the at least one support member (40A, 40B, 40C, 40D, 40E) may include an upper region with a recessed shoulder region on either side of the upper region. The cover member 30 seats down slightly into the body member 20, suspended around its periphery by at least one or more walls of the body member 20 in addition to being supported from below by the at least one support member (40A, 40B, 40C, 40D, 40E)s from the bottom end of the body member 20. This arrangement prevents side slippage of the cover member 30, yet allows for easy removal of the cover member 30 during the procedure if required to clear debris or for insertion, removal or cleaning of the drainage tube.

At least one filtration trap or more (50A, 50B, 50C, 50D) may be positioned proximal to the at least one drainage aperture (23A, 23B, 23C, 23D). of the body member 20. In a preferred embodiment, there are two filtration traps (50A, 50B, 50C, 50D). Of course, it is contemplated that there may be as many filtration traps (50A, 50B, 50C, 50D) as there are drainage apertures (23A, 23B, 23C, 23D). The filtration trap (50A, 50B, 50C, 50D) includes at least one wall surrounding perimeter of the drainage aperture (23A, 23B, 23C, 23D). In a preferred embodiment, the filtration trap (50A, 50B, 50C, 50D) includes two walls joined together at an angle. Of course, other shapes and positions of the filtration traps (50A, 50B, 50C, 50D) may be used in relation to the location of drainage aperture (23A, 23B, 23C, 23D).

To provide additional stability, the filtration traps (50A, 50B, 50C, 50D) have a lower profile than the side wall of the body member 20 to engage a top portion of the filtration trap (50A, 50B, 50C, 50D) with the cover member 30. In one embodiment, the filtration trap (50A, 50B, 50C, 50D) has a height approximately equal to the recessed shoulder region of the at least one support member (40A, 40B, 40C, 40D, 40E). The filtration trap (50A, 50B, 50C, 50D) may be permanently or releasably attached to the body member 20. For example, the filtration trap (50A, 50B, 50C, 50D) may be clipped or inserted into a grooved area defined within the bottom end 20A of the base member 20 which can be removed for cleaning when clogged with debris. The filtration traps (50A, 50B, 50C, 50D) may also be sized to help support the cover member 30.

Furthermore, an open cell material may be positioned proximal to the at least one cover aperture 33 or the at least one draining aperture in the platform to prevent clogging of the drainage tube 24 positioned within the at least one drainage aperture (23A, 23B, 23C, 23D) defined within the body member 20 for draining the fluid from the interior of the body member 20. For example, the open cell material may be a sponge material or mesh material for absorbing the fluid.

The at least one wall of the filtration traps (50A, 50B, 50C, 50D) defines at least one filtration aperture 51 for facilitating a flow of the fluid into the drainage aperture (23A, 23B, 23C, 23D) and drainage tube 24. The filtration trap (50A, 50B, 50C, 50D) is perforated by a series of multiple filtration apertures 51 large enough to allow for the free flow of blood, a brisk volume of irrigation fluid, and other fluids into the drainage aperture (23A, 23B, 23C, 23D) and drainage tube 24, yet small enough to prevent large pieces of tissue and other debris from falling into the drainage aperture (23A, 23B, 23C, 23D) and drainage tube 24 which may block or clog the drainage tube 24. As a result, the filtration traps (50A, 50B, 50C, 50D) creates a protected area, which may contain additional porous material to prevent debris from clogging the drainage tube 24. In another embodiment, the drainage tube 24 is attached to a suction pad that may or may not be covered for further protection in the protected area.

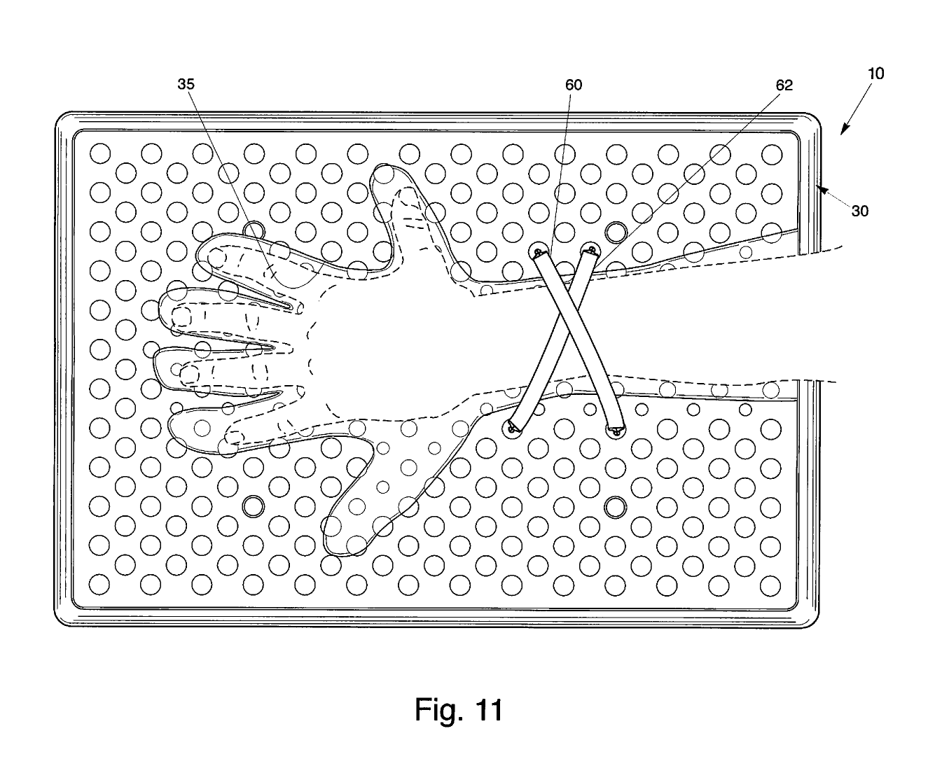

Referring to FIG. 11, a flexible strap member 60, 62 for securing the extremity to the platform may engage at least one cover aperture 33. The strap member 60, 62 includes a means for fastening at a proximal and distal ends of the strap member 60, 62 within the at least one cover aperture 33. The means for fastening at a proximal and distal ends of the strap members 60, 62 may include fasteners or other devices known in the art. For example, a hook may be used to secure the proximal or distal end of the strap member 60, 62 within the at least one cover aperture 33.

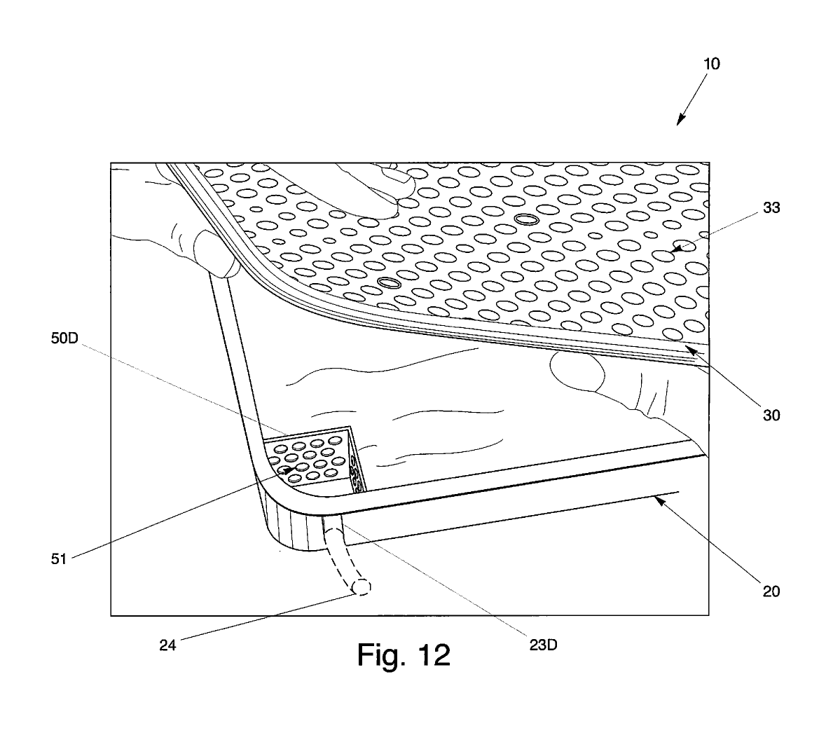

Referring to FIG. 12, the cover member 30 is sufficiently pliable for curling a portion of the cover member 30 proximal to the at least one drainage aperture (23A, 23B, 23C, 23D) to provide access to the interior of the body member 20 for removing clogging debris from the filtration trap (50A, 50B, 50C, 50D) and drainage tube 24 while maintaining the extremity (not shown) on the cover member 30. By locating the filtration trap (50A, 50B, 50C, 50D), at least one U-shaped drainage aperture (23A, 23B, 23C, 23D), and drainage tube 24 in corner areas of the platform 10, a physician can more easily remove the drainage tube 24 for cleaning and remove debris from the filtration traps (50A, 50B, 50C, 50D).

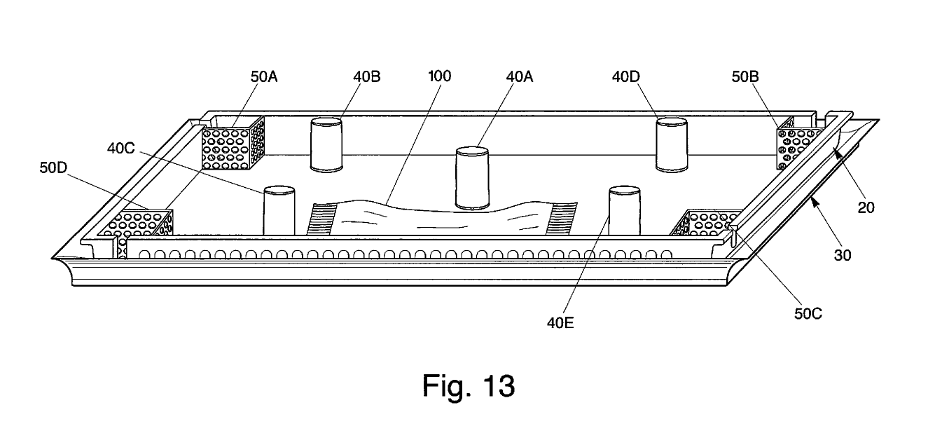

Referring to FIG. 13, a debridement surgical support kit for extremities of a human or mammal to assist a physician in treating an extremity may include the platform 10, as described herein, along with a surgery medical pack 100. The platform 10 including the body member 20 and the cover member 30. The cover member 30 nested with the body member 20 or the body member 20 nested within the cover member 30 to reduce the height of the platform 10 to a lower profile for more efficient transport. The surgery medical pack 100 including means for debridement and lavage of an extremity of a human known in the art such as surgical tools and other known surgical products for debridement and lavage. The surgery medical pack 100 is positioned within an interior of the nested platform to facilitate transport and reduce overall profile. A box having a low profile may be used for containing the nested platform and surgery medical pack 100 for purposes of transporting and storing the kit until required for surgery in a medical facility or storage area. In addition, one or more platforms 10 may be stackable within one another to further reduce consumption of valuable space within a medical facility, especially a surgical environment.

In operation, the extremity is positioned upon an upper surface of the cover member 30 to facilitate the process of debridement and lavage of extremities. Referring to FIG. 10, the extremity is seated within the predefined recessed area 35 for additional support. The strap members 60, 62 are then secured over the top of the extremity within the at least one cover aperture 33. During cleaning and debridement, the fluid travels through the at least one cover aperture 33 for temporary storage within an interior of the body member 20. Next, the fluid passes through the filtration trap (50A, 50B, 50C, 50D) to capture any debris which could potentially clog the drainage tube. The fluid then exits the body member 20 through the drainage tube 24 positioned within the drainage aperture (23A, 23B, 23C, 23D) for proper disposal of the fluid, after it exits the drainage tube 24, by a variety of methods known in the art. If the drainage tube 24 becomes clogged, a user curls or slightly opens a corner portion of the cover member 30 to reveal a corner area of the body member 20 where the filtration trap (50A, 50B, 50C, 50D) can be cleaned in place or removed for cleaning, the draining aperture (23A, 23B, 23C, 23D) can be cleaned, and the drainage tube 24 can be removed. Most importantly, the extremity may continue to be positioned on the cover member 30, if desired, during the cleaning and unclogging of the drainage tube 24, filtration trap (50A, 50B, 50C, 50D), and drainage aperture (23A, 23B, 23C, 23D).

By way of example only and without limiting the present disclosure to these specific dimensions, one embodiment of the debridement platform 10 may have the following dimensions. These dimensions are merely an example and in no way limit the scope of this subject technology. A multitude of different heights, sizes, or diameters may be used in conjunction with the present disclosure. The dimensions of the body member 20 is 1.5 inches tall×14 inches wide×22 inches long and the dimension of the lid member is approximately 0.25-0.50 inches tall×14 inches wide by 22 inches long. The dimension of the splash flange or guard 34 is 0.030 to 0.060 inches. The at least one support member (40A, 40B, 40C, 40D, 40E) is 2 inches in diameter and 1.25 inches-1.325 inches tall. Note, the at least one support member (40A, 40B, 40C, 40D, 40E) positioned in a central portion of the body member 20 is 1.25 inches tall with the other at least one support member (40A, 40B, 40C, 40D, 40E)s 1.325 inches tall to allow for a slight concavity of the cover member 30. At least one cover aperture 33 defined within the cover member 30 having a range of 0.125 diameter to 0.750 diameter.

The body member 20 and the cover member 30 can be made from metal, plastic or other suitably flexible or semi-flexible and fluid impermeable material which may be disinfected or sterilized if desired. Exemplary materials which may be used in construction of both are polyethylene, polycarbonate and stainless steel. Manufacturing methods include but are not limited to injection molding and stamping. The platform 10 is made of material selected from a variety of materials including polycarbonate, HDPE (high density polyethylene), low density polyethylene, plastic materials, biodegradable materials, regrind materials, and recycled plastic materials in combination or alone. Also, the platform 10 is injection molded using plastic materials to accommodate a wide range of extremity sizes and types at a lower cost.

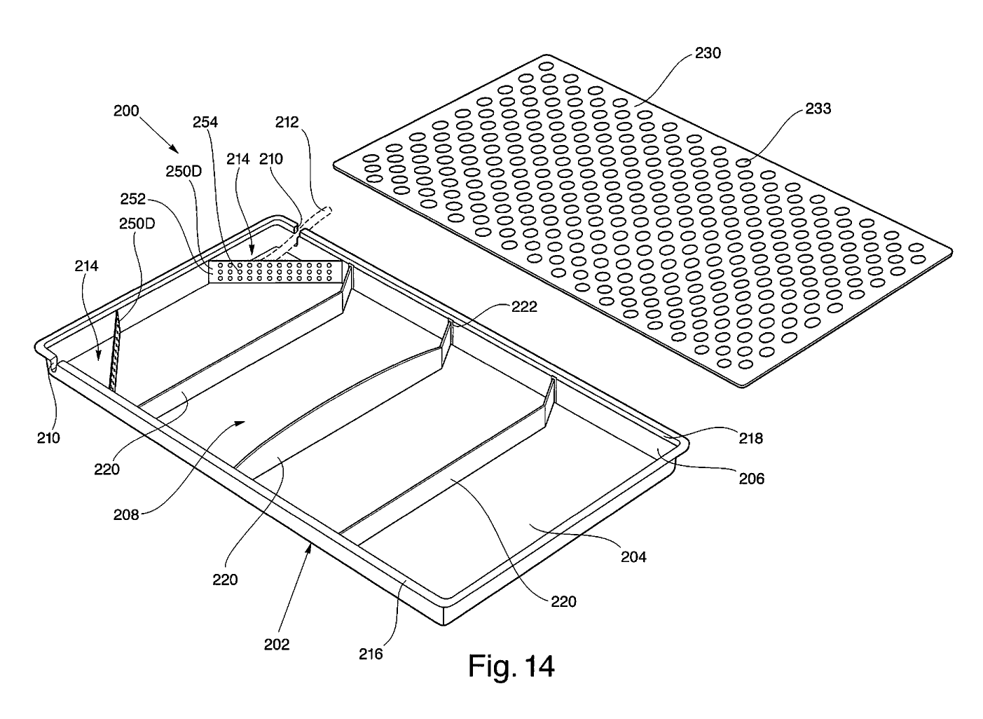

Referring now to FIG. 14, an exploded view of another debridement platform 200 in accordance with the subject technology is shown. Some elements are essentially the same as those of the foregoing embodiments and, thus, are not further described herein. The surgical support debridement platform 200 again provides support for extremities of a human to assist a physician in treating such extremity which incorporates the advantages and benefits of the above-mentioned platforms.

The platform 200 includes a body member 202 having a bottom 204 and a peripheral side wall 206. The body member 202 defines an interior volume 208 for temporarily containing fluid. In one embodiment, a depth of the body member 202 maybe 2-3 cm to define a low profile. Two drainage apertures 210 are defined in the side wall 206 of the body member 202 for draining fluid from the interior volume 208. One of the drainage apertures 210 is shown with a suction tube 212 drawn in dashed lines. Filtration traps 250D create a protected area 214 in which larger debris is kept away from the suction device (not shown). Preferably, the filtration traps 250D are a plate 252 with a plurality of throughholes 254. The suction tube 212 may attach to a suction pad or other mechanism to distribute the suction inlets throughout the protected area 214 or even into the interior 208 through a hole(s) in the filtrations trap(s).

Still referring to FIG. 14, the platform 200 includes a cover 230 defining a plurality of drainage holes 233. The cover 230 couples to the body member 202 to enclose the interior volume 208. The body member 202 includes an upper ridge 216 on top of the side wall 206 so that a shoulder 218 is formed. The cover 230 securely rests on the shoulder 218 and is restrained by the upper ridge 216. Preferably, the cover 230 snap fits onto the shoulder 218. The cover 230 may be concave, convex, wavy, channeled, grooved or any desired shape to conduct and/or collect fluid in a desirable location such as near an aperture 233 that corresponds to a protected area 214 or other desirable location in the interior 208.

The body member 202 also includes support members 220 to further support and position the cover 230 when assembled. As shown, the support members 220 are configured as wall members upstanding from the bottom 204 of the body member 202. The support members 220 may be parallel to one another and have a profile sufficient to support a cover 230 with an extremity (not shown) thereon. The support members 230 may, in another embodiment, include one or more wall members having inconsistent lengths and heights to provide stability to the cover 230 in a concave, convex, parabolic or other shape. As shown, the central support member 230 has a higher bowed upward top to create a slightly raised central portion when cover is 230 is flexible and snapped into place. In one embodiment, the filtration traps 250D act as support members for the cover 230.

In an alternative embodiment, the body member 202 does not form a shoulder 218 so that the support members 230 solely support the cover 230. The support members 230 may be removed. For example as shown, the side wall 206 forms opposing slots 222 into which the support members 230 and filtration traps 250D slide so that the support members 230 and filtration traps 250D are upstanding yet can be easily removed and laid flat.

With the support members 230 removed, the cover 230 may be stored against the bottom 204 along with the support members 230. As a result, a plurality of platforms 200 may be disassembled and stacked in a nested manner. In use, once assembled, the extremity is positioned upon a surface of the platform to facilitate the process of debridement and lavage of extremities.

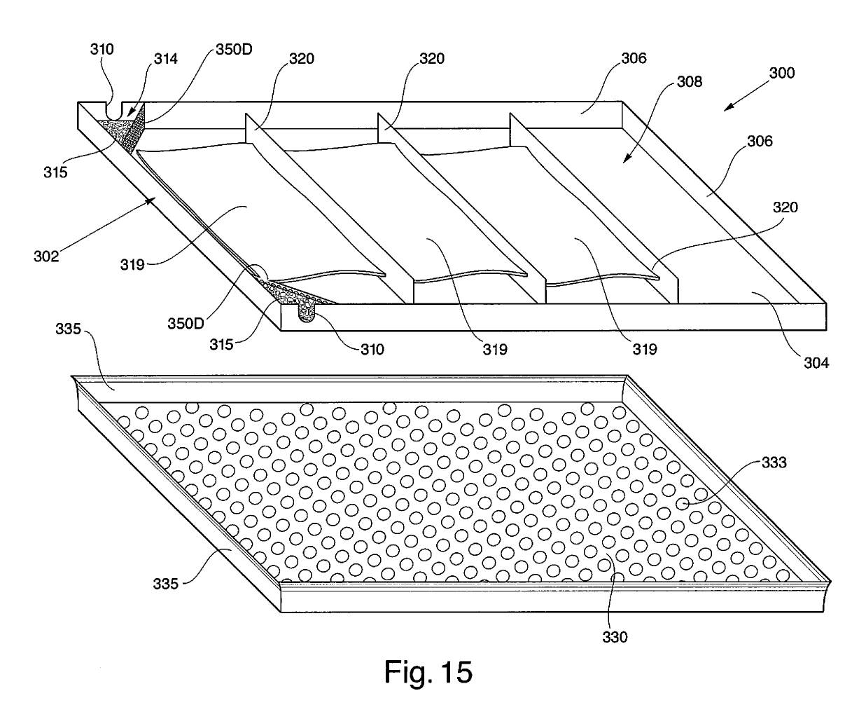

Referring now to FIG. 15, an exploded view of another debridement platform 300 in accordance with the subject technology is shown. Some elements are essentially the same as those of the foregoing embodiments and, thus, are not further described herein. The surgical support debridement platform 300 again provides support for extremities of a human to assist a physician in treating such extremity which incorporates the advantages and benefits of the above-mentioned platforms.

The platform 300 includes a body member 302 without any upper ridge so that a cover 330 may fit over the side wall 306. The cover 330 has apertures 333. The cover 330 includes a sidewall 335 so that, when assembled, the cover 330 is not inadvertently displaced from the body member 302. The cover 330 may also define slots (not shown) that align with apertures 310 to prevent interference with the suction tube(s).

The body member 302 includes one or more filter traps 350D. The protected or interior area 314 between the filter traps 350D and the section tube portals 310 may include a porous, or open cell material 315, such as foam to prevent clogging of the suction hose. For example, the porous material may be a variety of foam materials or composites of open cell or porous materials. It is also contemplated that the body member may include or not include filter traps 350D and instead use open cell or porous materials to catch debris from entering into the suction tube and thereby clogging. The filtration traps 350D may also be fabricated from a porous material. Although shown in the corners, it is also envisioned that the filter traps may be centrally located or in any other location and combination of locations.

The open cell or porous materials 315 may be dispersed or positioned about the interior 308 or bottom 304 of the body member 302 in a variety of configurations depending upon the extremity, size or other requirements. The open cell or porous material 315 may also act as a cushioning surface to prevent direct contact with a surface of the body member, cover, or both. Furthermore, it is contemplated that there may be additional open cell or porous material 319 sandwiched between the cover member 330 and the body member 302 to secure the open sell or porous material within the platform 300. Indeed, the sandwiched material 310 may support the cover member 330. In another embodiment, the foam or open cell material may be dispersed with antimicrobial or antibacterial coatings. For example, the antimicrobial coating may be antimicrobial silver. Supports 320 may also upstand from the interior surface 308. The supports 320 are approximately the same height as the sidewall 306. The cover 330 includes a sidewall 335. In another embodiment, the material 319 and the partitions 320 provide support to the cover member 330, which may have a shape such as convex or concave. In another embodiment, the body member 302 defines a port that the suction tube connects directly to. For example, the port may be in the bottom of the body member 302 such as a drainage hole formed in the center of the bottom and running through the bottom to form an edge or corner connector. The material 319 may be in the shape of sheets as shown, a plurality of stacked sheets, an over-sized ball that compresses and other variations as would be appreciated by those of ordinary skill in the art upon review of the subject disclosure.

Alternatively, the open cell or porous material may be located on any surface of the cover or body member. For example, the open cell material may be foam, more specifically polyurethane foam. Alternatively, materials other than open cell materials or porous materials may be used if the materials are configured to facilitate debridement and lavage of extremities. In one embodiment, foam or other suitable materials may be used within on a surface area of the body member, alone, or in combination with a second member. In another embodiment, foam or other suitable materials, may be used in combination with a basin or tray.

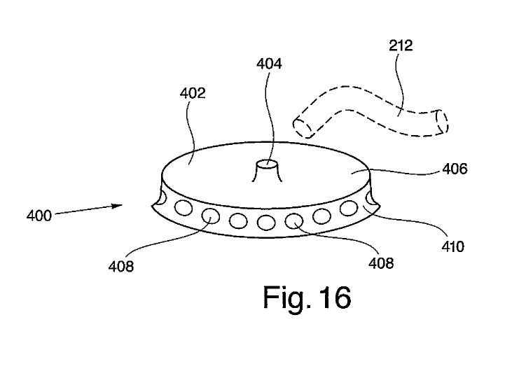

Referring now to FIG. 16, a perspective view of a suction pad 400 used in conjunction with a debridement platform is shown. The suction pad 400 is optional but may be used with any debridement platform. The suction pad 400 is shown with the suction hose 212 detached. The suction pad 400 may be placed in the body member to facilitate removal of liquid.

The suction pad 400 includes a central disc 402 with a central port 404 in an upper surface 406. The suction hose 212 connects to the central port 404 to provide vacuum thereto. The central port 404 is in fluid communication with a plurality of radial inlets 408 arranged in a sidewall 410 of the central disc 402.

For example, the suction pad 400 may be held within an interior 208, 308 of the body member 202, 302 by adhesives, fasteners, weights, or other securing mechanisms or methods. Typically, the central disc 402 would rest on the bottom 204, 304 of the body member 202, 302. The suction pad 400 may be made of solid or pliable materials, such as plastic.

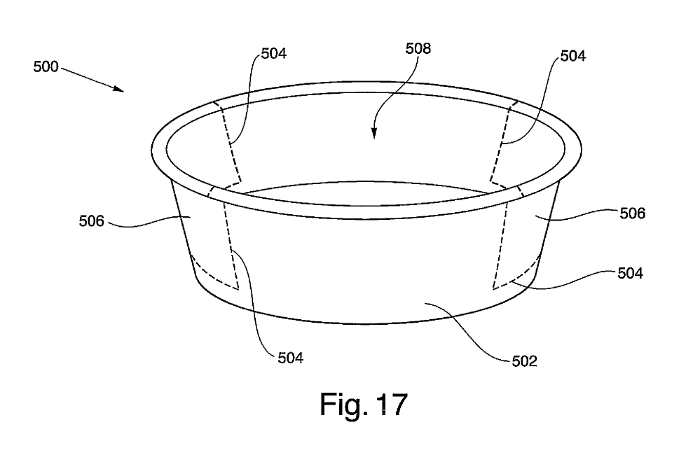

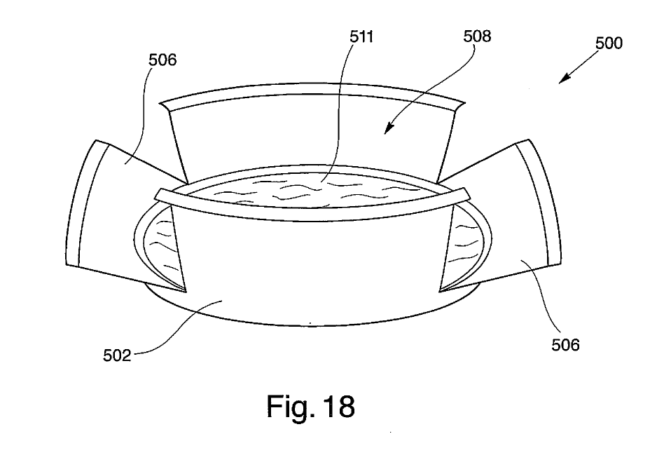

Referring now to FIGS. 17 and 18, perspective views of another embodiment of the debridement platform 500 in two positions are shown. The platform 500 has a body member 502 in the shape of a basin. Alternatively, the body member 502 may be combined with a second member or cover (not shown) to provide a platform. The second member, in one embodiment, may be another basin, tray, cover, or any of a number of other items used in conjunction with the body member to facilitate the debridement and lavage of an extremity.

The body member 502 defines perforations or cut-outs 504 to facilitate removal of one or more sectional flap areas 506 of the body member 502. For example, two opposing flap areas 506 may be removed or hinged away to provide a defined path to position an extremity within the body member 502. The sectional flap areas 506 may be hinged outward for removal or pivoting flat. Alternatively, the sectional flap areas 506 may be removed or turned inward to guide an extremity within an interior 508 of the body member 502. In another embodiment, the body member 502 may be pre-formed with the flap areas 506 removed in advance of use.

Furthermore, the interior 508 of the body member 502 includes an open cell or porous material 511, such as polyurethane foam. The foam 511 may be situated or dispersed about the surface of the body member 502. The body member 502 may also include a second member, such as a cover member, basin, tray, or other items to be used in conjunction with the body member to facilitate the debridement and lavage of an extremity. The second member may cover, nest, or otherwise engage the body member.

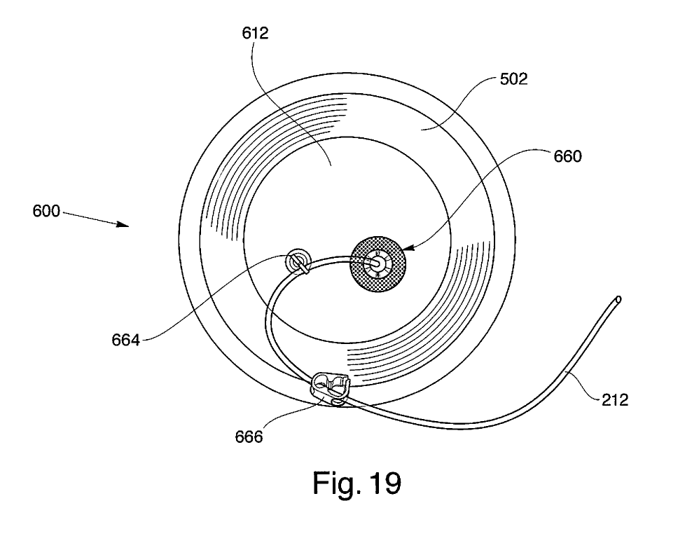

Referring now to FIG. 19, a top view of another debridement platform 600 is shown. Like reference numerals are used to show similar structure when possible. The platform 600 is again basin shaped but without perforations. The platform 600 includes a suction hose 212 attached to a filtration assembly 660. The filtration assembly 660 may be utilized with any embodiment shown herein. For example, the filtration assembly 660 may be used within the filtration trap of FIG. 12, in the protected area 314 of FIG. 15, and/or in the interior 308 of FIG. 15 etc.

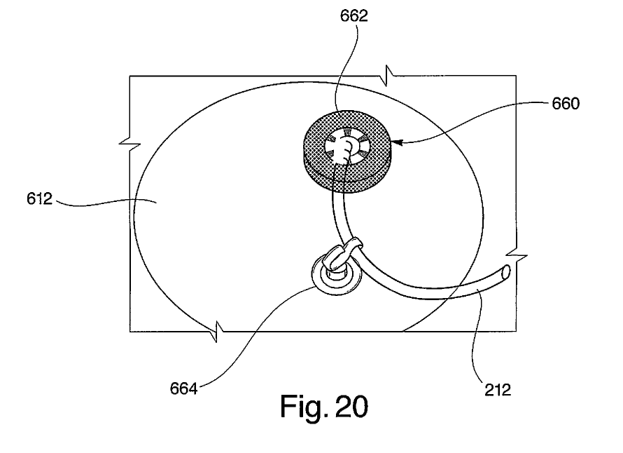

The suction hose, in one embodiment, is a standard operating room suction hose connected to a suction source or vacuum source (not shown), for use, for example, during surgical procedures for the removal of blood and tissue from an operation site. Referring additionally to FIG. 20, which is a partial top view of FIG. 19, the filtration assembly 660 is placed near, or more preferably, against a bottom 612 of the body member 602. The platform 600 may include a cover (not show) with drainage holes.

In operation, the extremity is positioned upon a surface of the platform 600 to facilitate the process of debridement and lavage of extremities. During the process of debridement and lavage of the extremities, the suction hose 212 removes fluid and tissue from the body member 602 without clogging due to the filtration assembly 660.

The filtration assembly 660 may include the suction pad 400 shown in FIG. 16 with a porous material 662 covering the central disc 406. In another embodiment, the central disc 406 is an imbedded flexible plastic or silicone support pad to provide support or rigidity. The filtration assembly 660 is configured to be attached, removably or permanently, to any standard operating room suction hose. The porous material 662 is positioned on the suction pad 400 to prevent the suction hose 212 from clogging when the suction source is in operation. The porous material 662 provides filtration of tissue. For example, without limitation, the porous material is foam, more specifically a polyurethane foam. The size and dimension of the porous material and the number of suction hoses with filtration assemblies can be varied according to need. The filtration assembly 600 may be also modified into many shapes for additional suction devices, such as a wand, both for draining fluid collection from the body members as well as for directly suctioning wounds. Of course, the filtration component may also be used by itself with the suction hose and without the body member.

In one embodiment, the suction hose 212 is connected to the body member 502 using a suction cup assembly 664. The suction cup assembly 664 is particularly useful for holding smaller suction hoses in place on the body member 502. Optionally or additionally, the suction hose 212 may be connected to the body member 502 using an on/off clamp 666. Of course, it is contemplated that the suction hose 212 may be connected to the body member 502 using a variety of fasteners or other types of fastening or securing means.

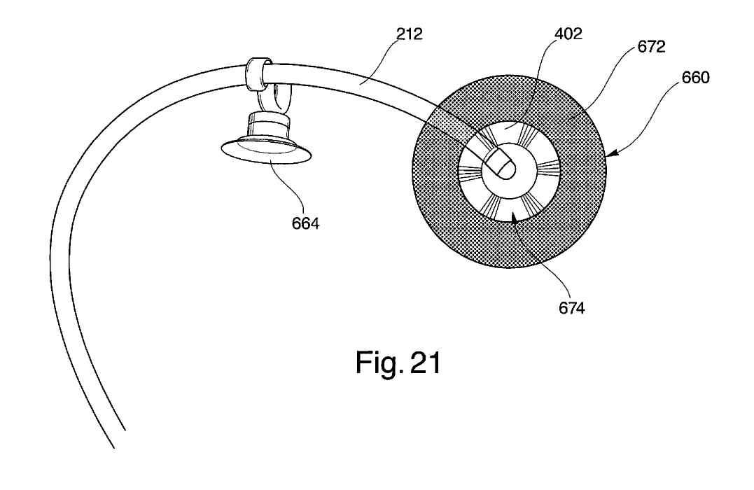

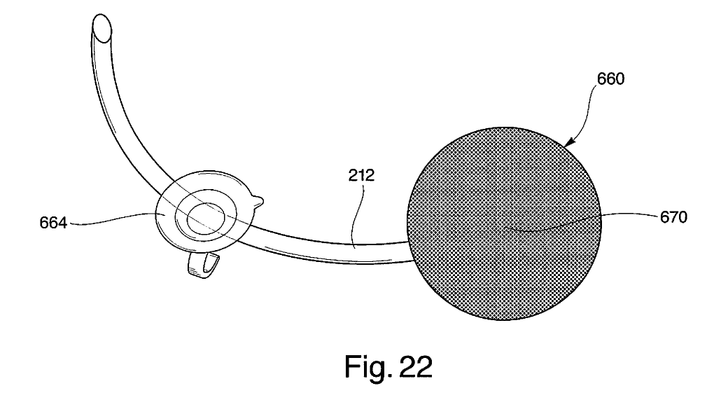

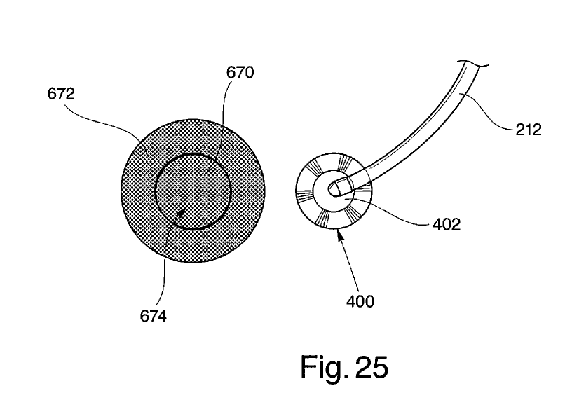

Referring now to FIGS. 21 and 22, top and bottom perspective views of the filtration assembly 660 are shown. As best seen in the exploded view of FIG. 25, the porous material 662 is similarly disc-shaped like the suction pad 400. The porous material 662 preferably includes a solid bottom 670 with a top 672 that defines an opening 674 for insertion of the central disc 402 therein.

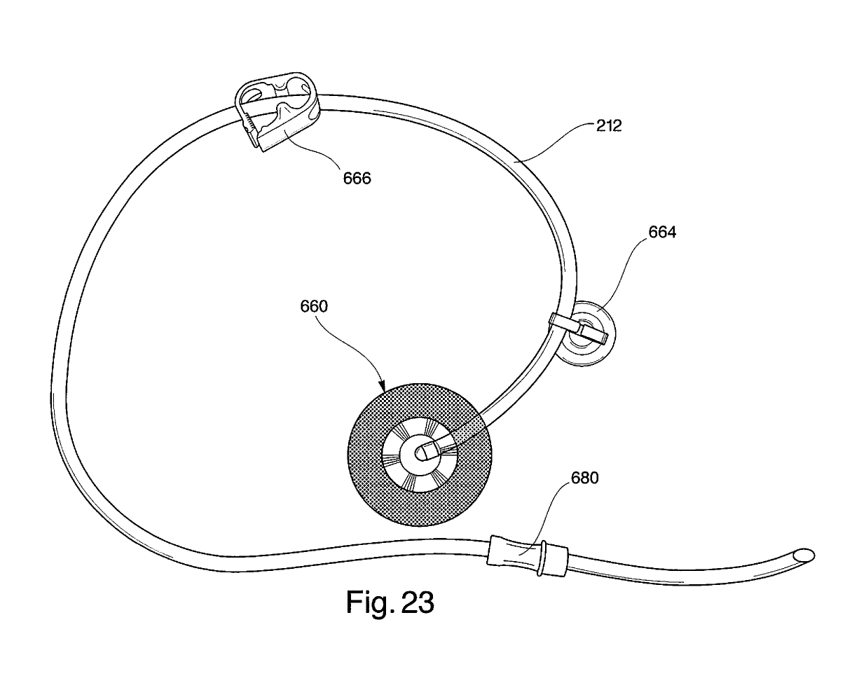

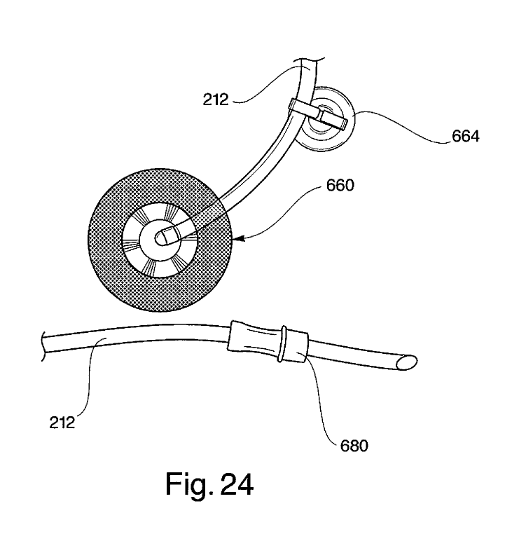

Referring now to FIGS. 23 and 24, top views of the suction hose 212 and filtration assembly 660 of FIG. 19 having an additional filter component 680 are shown. The additional filter component 680 is connected in-line with the suction hose 212. Preferably, the filter component 680 is easily replaced in the event of clogging and provides additional removal of debris in the fluid passing therethrough.

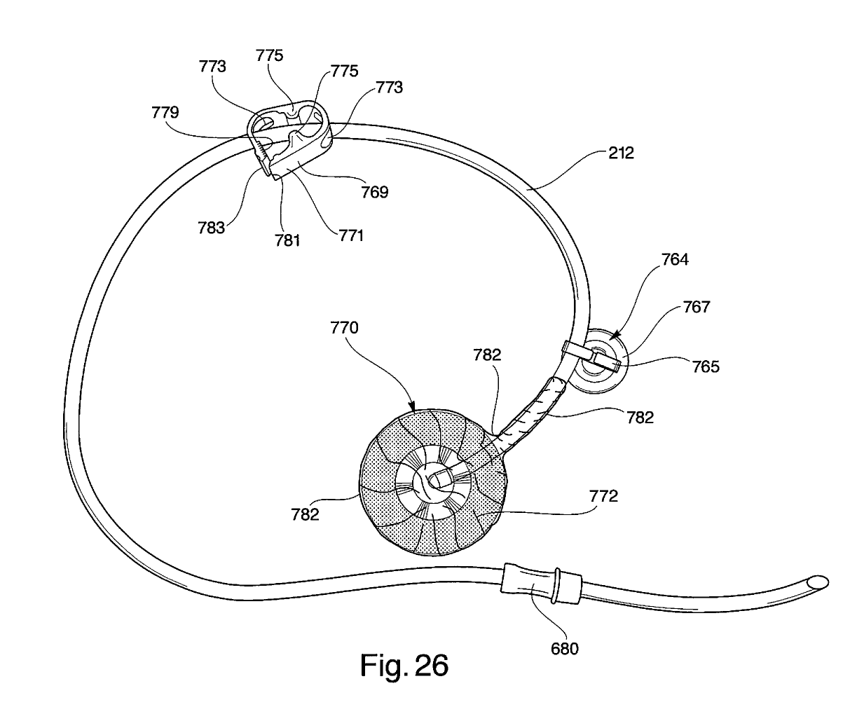

Referring now to FIG. 26, another filtration assembly 770 is shown. Like reference numerals are used to describe similar structure when possible. The filtration assembly 770 is similar to the filtration assembly 670. The filtration assembly 770 additionally includes a wrapping layer 782 on the top 772 of the porous material 762 of the pad 400. The wrapping layer 782 prevents debris from passing through the opening 774 and clogging the filtration assembly 770. The wrapping layer 782 may be semi-porous or a solid barrier like heavy duty plastic film. The wrapping layer 782 may include an adhesive backing for liquid tight sealing to the top 772 of the porous material 762. An elastic, clamp, clip, pins or other fastener (not shown) may also be used to secure the wrapping layer 782 in place.

A suction cup assembly 764 includes a clip 765 for attaching a suction cup 767 to the suction tube 212. As a result, the suction tube 212 can be secured to a desired location for proper routing and the filtration assembly 770 can be located at any desired location in the interior such as in a protected area or on a sidewall even. As many suction cup assemblies 764 may be provided as needed. The suction tube 212 also has a stop clip 769 mounted thereon. The stop clip 769 allows a user to selectively turn off the suction near or even inside the debridement platform. The stop clip 769 includes a band-shaped body 771 formed into an oval so that two opposing apertures 773 are formed for sliding onto the suction tube 212. Opposing bumps 775 on the body 771 interact to occlude the suction tube 212 when the clip 769 is closed. The closure mechanism 777 includes a plurality of angled teeth 779 that interact with a beveled end 781 so that as the closure mechanism 777 is squeezed, the beveled end 781 is captured in the teeth 779. To release the end 781, the opposing end 783 that carried the teeth 779 is pulled back.

In another embodiment, a debridement surgical support kit is provided for extremities of a human to assist a physician in treating an extremity. The platform, in one embodiment, includes a platform including a first member, a suction hose having a filtration component for draining said fluid from the interior of said first member; and a surgery medical pack including means for debridement and lavage of an extremity of a human, said surgery medical pack positioned within an interior of said platform.

It would be appreciated by those skilled in the art that various changes and modifications can be made to the illustrated embodiments without departing from the spirit of the subject technology. All such modifications and changes are intended to be covered by the appended claims.

What is claimed is:

1. A surgical support debridement platform for supporting an extremity of a patient to assist a physician in treating the extremity, the platform comprising:
a body member including a bottom and at least one sidewall surrounding the bottom to define an interior for containing fluid and at least one drainage aperture for draining said fluid from the interior;
a cover including an upper surface configured to engage an extremity, the cover being releasably engaged to the body member and defining at least one aperture for facilitating flow of the fluid through the cover and into the interior; and
a suction device at least partially in the interior for removing the fluid therefrom.

2. The platform of claim 1, wherein the suction device includes: a distal circular member defining a port in fluid communication with a plurality of radial inlets; a covering on the distal circular member to prevent clogging of the plurality of radial inlets; a tube in fluid communication with the port for providing vacuum thereto; and a clip with a suction cup attached to the tube for selectively securing the distal circular member at a desired location in the interior.

3. The platform of claim 2, further comprising a wrapping layer on the covering to prevent debris from coming between the covering and the distal circular member, wherein the covering is a porous material and the at least one sidewall defines a slot through which the tube passes.

4. The platform of claim 1, wherein the upper surface of said cover member defining an overall concave shape to prevent pooling of a fluid about an extremity.

5. The platform of claim 1, wherein the cover releasably engages the body member so that the body member can nest within the cover.

6. The platform of claim 1, wherein the cover defines a recessed extremity area for providing further stability to the extremity while seating on the cover and the at least one drainage aperture generally defines a U-shape, wherein the at least one drainage aperture is positioned proximal to a corner area of said body member, said drainage aperture positioned in a lower portion of said sidewall substantially proximal to the bottom end of said body member, and the cover is sufficiently pliable for curling upward proximal to said at least one drainage aperture to provide access to the interior for removing clogging debris and drainage tube while maintaining the extremity on the cover.

7. The platform of claim 1, further comprising a filtration trap including at least one wall in the interior to define a protected area surrounding the at least one drainage aperture.

8. The platform of claim 7, wherein the at least one wall of the filtration trap is a porous material and the protected area includes a porous material for keeping debris away from a suction tube extending through the at least one drainage aperture.

9. The platform of claim 1, further comprising a plurality of support members upstanding from the bottom for supporting the cover, wherein the cover has a concave shape and the plurality of support members have different heights to provide stability to the cover and maintain the concave shape.

10. The platform of claim 9, wherein the at plurality of support members define at least two areas protected from debris for receiving a porous material in the protected areas.

11. The platform of claim 1, wherein the suction device comprises a suction pad having: a central disc with an upper circular surface defining a central port for connection to a suction hose, wherein the central disc has a sidewall defining a plurality of radial peripheral inlet ports in fluid communication with the central port; a porous material covering the plurality of inlet ports; and a wrapping covering a portion of the porous material for preventing debris from coming between the porous material and the central disc.

12. A surgical support debridement platform for extremities of a human to assist a physician in treating such extremity comprising:
a body member including a bottom and a side wall surrounding the bottom to define an interior volume for temporarily containing fluid;
a filter trap coupled to the body member to define a suction area within the interior, wherein the filter trap includes a partition wall defining a plurality of apertures; and
a filtration component in the suction area for preventing clogging of a suction hose within the body member for draining the fluid from the suction area.

13. The platform of claim 12, further comprising at least one support member upstanding from the bottom to support the cover and the cover has a concave shape.

14. The platform of claim 12, wherein the at least one support member defines an area protected from debris for receiving the filtration assembly; and further comprising a porous material in the protected area.

15. The platform of claim 14, wherein the filtration assembly includes a a central disc having an upper surface opposing a lower surface with a sidewall extending between the upper and lower surfaces, the upper surface defining a central port for connection to the suction hose and the sidewall a plurality of radial inlets in fluid communication with the central port; and a porous material covering at least the plurality of radial inlets.

16. A suction pad for use with a debridement platform comprising:
a central disc having an upper surface opposing a lower surface with a sidewall extending between the upper and lower surfaces, the upper surface defining a central port for connection to a suction hose and the sidewall a plurality of radial inlets in fluid communication with the central port; and
a porous material covering at least the plurality of radial inlets.

17. A suction pad as recited in claim 16, wherein the porous material is similarly disc-shaped like the central disc and includes a solid bottom opposing a top that defines an opening for passage of the central disc therethrough.

18. A suction pad as recited in claim 17, further comprising a wrapping layer for covering the opening for preventing debris from entering the opening.
